(19) United States
(12) Patent Application Publication
(10) Pub. No.: US 2010/0098768 A1
(43) Pub. Date: Apr. 22, 2010

(54) Method of neuroprotection from oxidant injury using metal oxide nanoparticles

(75) Inventors: Emanuela Silvana Andreescu, Potsdam, NY (US); James C. Leiter, Woodstock, VT (US); Joseph S. Erlichman, Canton, NY (US)

Correspondence Address: BOND, SCHOENECK & KING, PLLC, ONE LINCOLN CENTER, SYRACUSE, NY, 13202-1355, US

(73) Assignee: CLARKSON UNIVERSITY, Potsdam, NY (US)

(21) Appl. No.: 12/580,859

(22) Filed: Oct. 16, 2009

Related U.S. Application Data
(60) Provisional application No. 61/105,926, filed on Oct. 16, 2008.

(51) Int. Cl. A61K 9/14; A61K 33/24; A61P 39/00; A61P 9/10

(52) U.S. Cl. 424/489; 424/649; 424/617

(57) ABSTRACT

A metal oxide nanoparticle composition including a cerium oxide nanoparticle and a metal adapted to enhance the neuroprotective activity of the cerium oxide nanoparticle. The metal can include noble metals such as platinum, and rare earth metals such as gadolinium, samarium, titanium, yttrium, zirconium, and a combination thereof Another metal oxide nanoparticle composition including a cerium oxide nanoparticle and a surface modifier, such as polyethylene oxide, polyethylene imine, dextran, polylactic acid, chitosan, alginate, and a combination thereof is provided. A method of using the metal oxide nanoparticle compositions as neuroprotective agents for the inactivation of reactive oxygen species in nervous tissues is also provided. More specifically, a neuroprotective method using the metal oxides such as ceria, yttria, or mixed ceria and yttria (or any of the other referenced metal oxide nanoparticle compositions) before, during, or after an ischemic event.

### RELATED APPLICATION DATA

The present application claims priority to U.S. provisional patent application number 61/105,926, filed Oct. 16, 2008; all of the foregoing patent-related documents are hereby incorporated by reference herein in their respective entireties.

### BACKGROUND OF THE INVENTION

1. Field of the Invention

The present invention relates to the use of nanoparticles of metal oxide compositions to protect nervous tissues before, during, and after oxidant injury, and, more particularly, to the use of cerium, yttrium, and mixed cerium and yttrium based metal oxides to protect nervous tissues from reactive oxygen species both acutely and chronically.

2. Description of the Related Art

Ischemia is a reduction of blood flow to an organ or area of the body caused by blockage or constriction of blood vessels typically resulting from, among other things, artherosclerosis, thromboembolism, hypotension, tachycardia, or sickle cell disease. The reduction of blood flow prevents the adequate delivery of oxygen to cells and results in hypoxic or anoxic tissues. This prolonged oxygen deprivation typically results in cellular damage and cell death.

Nervous tissues are among the most sensitive tissues to reduced oxygen supply. Oxygen toxicity in the central nervous system may also occur when oxygen levels are excessive. Oxidant injury resulting from either oxygen excess or oxygen deficiency is caused by nitric oxide as well as peryoxynitrite, hydroxyl, and superoxide radicals. These agents are toxic to neurons and glia and contribute to neuro-glial degeneration as a result of ischemia, traumatic brain injury, and a variety of degenerative diseases such as amyotrophic lateral sclerosis. During an ischemic episode, an ischemic cascade is triggered that can cause irreversible death of nervous tissue. This may occur as a result of vasculature occlusion or inadequate vascular control, such as in periventricular leukomalacia, which may lead to cerebral palsy. Shortly after a neuron is deprived of oxygen, membrane transport systems slow and the neuron becomes depolarized. This results in the release of excitatory neurotransmitters which stimulate calcium and sodium influx. The increased intracellular concentration of calcium interferes with metabolic processes, activates degradative enzymes, and causes the formation of free radicals. These effects ultimately cause extensive neuronal damage and can lead to cell death.

Free radicals are ions or small molecules with unpaired electrons in the valence, or outermost, shell. Free radicals are formed when a covalent bond between two atoms is broken and one electron remains with each atom, or by oxidation or reduction of an atom or molecule, or by ionizing radiation. Free radicals such as hydroxyl radicals, nitric oxide, and superoxide are produced by cells and tissues as by-products of important metabolic processes (both normal and pathological) and are believed to play important roles in the body including serving as intracellular signaling molecules or ions, regulating programmed cell death, and participating in the normal functioning of the immune system. Mitochondria, for example, generate free radicals as part of a normal series of steps in which carbon-based fuels (glucose, fats and proteins) are oxidized by oxygen. But many pathological processes, such as inflammation, ischemia, and reperfusion, also generate free radicals. Humans are also continuously exposed to free radicals in the environment as a result of pollutants, exposure to ultraviolet light, and ozone, for example.

As a result of the unpaired electron(s) in the valence shell, free radicals are highly reactive and tend to participate in chemical reactions that generate additional free radicals with lower chemical reactivity. Superoxide and nitric oxide radicals, for example, may combine to form peroxynitrite, which is a potent oxidizing and nitrating agent. Although nitrification of amino acids such as tyrosine also plays an important role in cell signaling, elevated concentrations of peroxynitrite can lead to increased nitrosylation of proteins which in turn may induce apoptosis and cell death. Thus, while free radicals have a role in normal cell processes, under pathological conditions the production of free radicals and peroxynitrite can result in a combined attack on a variety of signaling molecules as well as cellular structural elements (particularly lipids), leading to the disruption of normal cellular processes and eventually cell death.

To prevent damage caused by free radicals, the body possesses a variety of detoxification and regulation mechanisms, including enzymes such as superoxide dismutase, glutathione peroxidase, and catalase which convert free radicals to less toxic substances in the presence of appropriate substrates, and chemical compounds that can donate an electron to the free radical in order to reduce its reactivity. Despite these protective mechanisms, free radicals arising from either endogenous production or exogenous sources can quickly exceed the regulatory capacity of the cell.

Since the mechanisms of free radical formation are ubiquitous, a wide variety of diseases are thought to arise from excess free radical formation and reactivity. For example, free radicals are thought to play a role in the normal aging process (as suggested by the life-extending properties of antioxidant compounds such as resveratrol), cancer formation, and atherosclerosis. In the brain, excess free radical formation may contribute to amyotrophic lateral sclerosis, Alzheimer's disease, stroke, ischemic brain injury, traumatic brain injury, and the degradation of dopaminergic neurons in Parkinson's disease, among others.

During ischemia, large amounts of a variety of free radicals are produced, including reactive oxygen species (“ROS”) such as superoxide and its derivatives hydroxide and hydrogen peroxide, peroxynitrite, nitric oxide, and nitrogen oxide, among others. The sudden increase in ROS production quickly overwhelms the cell's ability to neutralize the free radicals and results in extensive damage to the cell, including damage to the cellular DNA. The rapid increase in ROS production is thought to be the primary cause of ischemic injury.

Chronic exposure to low grade oxidant injury may also cause neurodegenerative damage. This is thought to occur in a variety of neurodegenerative diseases such as ALS, Alzheimer's disease and Parkinson's disease, among others. A similar biochemical cascade leading to generation of excess free radicals may occur in these entities and in the setting of traumatic brain injury. Though less dramatic than an acute ischemic event, low grade release of excess oxidants may nonetheless lead to profound neuronal loss over time.

Reperfusion, the restoration of blood flow following an ischemic episode, can also be extremely damaging to tissues. The increase in intracellular oxygen concentrations following reperfusion often results in increased production of ROS, causing greater cellular damage and potentially leading to cell death. The damage caused by the restoration of blood flow after an ischemic event is called reperfusion injury.

To prevent ischemic injury, low grade oxidant injury, and reperfusion injury, researchers have studied a number of approaches intended to inhibit one or more pathways of the ischemic cascade. These approaches are termed ‘neuroprotection’ and include: ion channel blockers that prevent the passage of calcium, sodium, and potassium ions; neurotransmitter antagonists that prevent the activity of neurotransmitters such as glutamate, gamma-aminobutyric acid, and serotonin; and free radical scavengers such as antioxidants to find and neutralize free radicals. Antioxidants such as vitamin E, vitamin C, and carotenoids have all been used to treat a variety of ‘oxidant injury’ diseases.

The relevant art is described in further detail in the following references, all of which are hereby incorporated by reference: A. S. Karokoti, N. A. Monteiro-Riviere, R. Aggarwal, J. P. Davis, R. J. Narayan, JOM Journal of the Minerals, Metals and Materials Society, 2008, 60, 33; G. R. Bamwenda, H. Arakawa, J. Mol. Catal. A. Chemical, 2000, 161, 10113; S. V. Manorama, N. Izu, W. Shin, I. Matsubara, N. Murayama, Sens. Actuat. B, 2003, 89, 299; S. S. Lin, C. L. Chen, D. J. Chang, C. C. Chen, Water Res., 2002, 36, 3009; S. Hamoudi, F. Larachi, G. Cerrella, M. Cassanello, Ind. Eng. Chem. Res., 1998, 37, 3561; C. Korsvik, S. Patil, S. Seal, W. T. Self, Chem. Commun., 2007, 1056; P. Dutta, S. Pal, M. S. Seehra, Chem. Mater., 2006, 18, 5144; M. Das, S. Pati, N. Bhargava, J. F. Kang, L. M. Riedel, S. Seal, J. J. Hickman, Biomaterials, 2007, 28, 1918; R. W. Tarnuzzer, J. Colon, S. Seal, Nano. Lett., 2005, 5, 2573; J. F. McGinnis, J. Chen, L. Wong, S. Sezate, S. Seal, S. Patil, U.S. Pat. No. 7,347,987, Mar. 25, 2008; A. Y. Abramov, A. Scorziello, M. R. Duchen, J. Neurosci., 2007, 27, 1129; F. Stoffels, F. Lohofener, M. Beisenhirtz, F. Lisdat, R. Biittemeyer, Microsurgery, 2007, 27, 565; R. Biittemeyer, A. W. Philipp, J. W. Mall, B. X. Ge, F. W. Scheller, F. Lisdat, Microsurgery, 2002, 22, 108; B. A. Rzigalinski, I. Danelisen, E. T. Strawn, A. A. Cohen, C. Liang, C. in Tissue, Cell and Organ Engineering (Ed. S. S. Challa and R. Kumar), Wiley-VCH, Weinheim, Germany, 2006, Vol. 9; D. Schubert, R. Dargusch, J. Raitano, S. W. Chan, Biochemical and Biophysical Research Communications, 2006, 342, 86.

Description Of the Related Art Section Disclaimer: To the extent that specific publications are discussed/listed above in this Description of the Related Art Section, these discussions/listing should not be taken as an admission that the discussed/listed publications (for example, published patents) are prior art for patent law purposes. For example, some or all of the discussed/listed publications may not be sufficiently early in time, may not reflect subject matter developed early enough in time and/or may not be sufficiently enabling so as to amount to prior art for patent law purposes. To the extent that specific publications are discussed/listed above in this Description of the Related Art Section, they are all hereby incorporated by reference into this document in their respective entirety(ies).

### BRIEF SUMMARY OF THE INVENTION

The present invention recognizes that there are potential problems and/or disadvantages in the above-discussed methods of treating or preventing cellular damage caused by free radicals. One potential problem is that there are, to date, no antioxidant agents with proven efficacy in neurological diseases. Antioxidants such as vitamin E, vitamin C, and the carotenoids have proven unsuccessful (with the possible exception of vitamin E as a preventative therapy in atherosclerotic heart disease). These agents are believed to have failed for a variety of reasons. First, their antioxidant power is limited. Second, they have difficulty penetrating the blood brain bather and gaining access to the site of free radical formation in the brain. Third, the production of free radicals occurs rapidly and early in the disease process and administration of antioxidant agents after the initial injury is ineffective. Since ions enter an oxygen-deprived cell and cause the release of neurotransmitters very early in the ischemic cascade, ion channel blockers and neurotransmitter antagonists are typically only effective if administered before or quickly after the ischemia begins, an often difficult or impossible target to meet. Finally, neuroprotective agents tried in the past have had a relatively short duration of effect. As a result of these limitations, there is still a need for effective and easily-administered neuroprotective agents that can be used to prevent and treat ischemic injury in a relevant timeframe. Various embodiments of the present invention may be advantageous in that they may solve or reduce one or more of the potential problems and/or disadvantages discussed in this paragraph.

It is therefore a principal object and advantage of the present invention to provide a method to protect neuronal tissues against ischemic injury and reperfusion injury, neurodegeneration, traumatic brain injury, and hyperoxic brain injury caused by reactive oxygen species.

It is another object and advantage of the present invention to provide a method for protecting neuronal tissues against reactive oxygen species in a medically-treatable timeframe.

It is a further object and advantage of the present invention to provide an agent that detoxifies free radicals that can have wide applicability in variety of neurological diseases and which may be used either preventatively or for treatment of chronic degenerative illnesses.

It is yet another object and advantage of the present invention to provide a method for protecting neuronal tissues against reactive oxygen species using an agent that is easily administered, can cross the blood/brain bather, and is readily taken up by cells. This can be achieved by modifying the surface characteristics of certain nanoparticles, as described infra, to alter lipophilicity, aggregation, and other physical characteristics or by doping the nanoparticle with other metals.

### SUMMARY OF THE INVENTION

In accordance with the foregoing objects and advantages, the present invention provides a method of using novel nanoparticles of metal oxides that provide more potent antioxidant activity than previous conventional antioxidant therapy. More specifically, a neuroprotective method using nanoparticle compositions as neuroprotective agents for the inactivation of reactive oxygen species in nervous tissue is provided.

Cerium and yttrium, for example, are metal elements that have antioxidant properties in certain states. Cerium is a lanthanide metal element which can exist in two states, Ce³⁺ and Ce⁴⁺, which are interchangeable in a reduction-oxidation environment. Cerium oxide, which is also called ceria (molecular formula CeO₂), possesses unique auto-catalytic reduction-oxidation properties which have been attributed to the highly mobile lattice oxygen present at its surface as well as a large diffusion coefficient of the oxygen vacancy that facilitates the conversion of Ce⁴⁺ and Ce³⁺ between valence states and thus allows oxygen to be stored in or released from its crystalline structure. Yttrium is a trivalent transition group 3 element with strong similarity to the lanthanoids. Yttrium oxide, also known as yttria (molecular formula Y₂O₃) is similar to ceria and has catalytic reduction-oxidation properties that allow it to act as a catalyst to mimic the reduction-oxidation characteristics of enzymes such as superoxide dismutase.

Cerium-oxide based nanoparticles possess a number of advantages over other antioxidants. First, these nanoparticles act as catalysts to mimic superoxide dismutase activity. Second, the nanoparticles are not consumed as they detoxify free radicals because they reconstitute their catalytic function by moving spontaneously between oxidized and reduced states. As a result they remain resident in the tissue and active for extended periods of time. Third, when administered systemically the nanoparticles cross the blood brain bather, thereby allowing for the treatment of neural damage or disease. Other advantages of the embodiments of the present invention are presented herein or will be apparent to one skilled in the art.

The antioxidant activity of cerium-oxide nanoparticles can be enhanced if they are in contact with noble metals such as platinum, among others, or ‘doped’ with rare earth metals such as yttrium, gadolinium, samarium, zirconium, or titanium, among others. These added metals are believed to facilitate the transfer of oxygen from the bulk material to the surface and vice-versa. The Examples below discuss the assessment of the antioxidant potency of these cerium congeners using in vitro tests as well as the brain slice model of ischemia.

The nanoparticles of metal oxide can also be modified with surface modifiers such as polyethylene oxide, polyethylene imine, dextran, polylactic acid, chitosan, or alginate, among others to modify characteristics such as surface charge, biocompatibility, cellular uptake, and in vivo circulation time. These specialized coatings may also give the nanoparticles tissue-specific targeting properties or facilitate administration by preventing clumping and agglutination.

Since the cerium-oxide nanoparticles appear to be non-toxic and remain active in tissues for extended periods of time, they can be administered either preventatively or at an early stage of a chronic disease process. Traditionally, neuroprotective agents are administered immediately before or immediately after the onset of injury. As a result, a long-term preventative agent represents a major improvement in the field. For example, soldiers at risk of traumatic brain injury might be given prophylactic nanoparticle injections weeks before exposure to combat. The injections can be repeated every 4-6 weeks as a booster, but the neuroprotective effect will likely linger for weeks to months after initial therapy. The therapy can be given intravenously and the dose can be in the range of 0.5 to 1 μM/kg, for example. While it may be necessary to give a series of loading doses, stable ongoing antioxidant therapy is likely to require single IV injections approximately every 4-6 weeks.

The Examples below describe a number of studies which explore and demonstrate the utility of metal oxide nanoparticle compositions and some of its congeners in treating or preventing oxidant injury. For example, a study was completed regarding the neuroprotective effect of ceria nanoparticles in a brain slice model of hippocampal ischemia. The results showed that cerium-oxide nanoparticles suppressed cell death, reduced the formation of free radicals and reduced nitrosylation of proteins compared to untreated brain slices during simulated brain ischemia as discussed in Example 3. Yet another study examined the increased antioxidant activity of yttrium-doped ceria and platinum-doped ceria in the brain slice model, as discussed in Example 5.

In summary, metal oxide nanoparticles of an embodiment of the present invention catalyze the detoxification of free radicals. It seems likely that these nanoparticles will have unusually potent effects in a variety of neurological diseases in which excess free radical formation is thought to play a role. These range from relatively rare diseases such as ALS, to more common conditions such as strokes, traumatic brain injury, Parkinson's disease and Alzheimer's disease as well as the ubiquitous normal processes of aging.

### DETAILED DESCRIPTION OF THE INVENTION

Since ceria and yttria release oxygen and undergo rapid, reversible reduction/oxidation reactions, the metal oxides serve as a reduction/oxidation cycling agent that do not themselves generate free radicals in the process. Electron shuffling in the lattice along with the electron vacancies provides the reduction/oxidation potential for free radical scavenging. The metal oxides are not consumed in this reaction and remain active for extended periods of time.

In one embodiment of the current invention, nanoparticles of ceria and/or yttria are introduced post-ischemia at a time when ROS production is high. In order to determine the neuroprotective capabilities of ceria and yttria, the compounds were added to animal models at specific time-points following ischemia, as described in the Examples below. Specifically, ceria was applied since the compound has previously been shown to be a potent free radical scavenger in cell culture systems.

Advantages of the invention are illustrated by the following Examples. However, the particular materials, amounts thereof, products, physical testing equipment and/or machines recited in these examples, as well as other conditions and details, are to be interpreted to apply broadly in the art and should not be construed to unduly restrict or limit the invention in any way.

### Example 1

This Example describes the examination of the brain cell uptake of fluorescently labelled ceria nanoparticles. To facilitate crossing of the blood/brain bather and the rapid uptake by cells, ceria was applied in the form of roughly 10 nanometer nanoparticles. In one set of experiments the metal oxide nanoparticles were covalently attached to a fluorescent label before being applied to the animal model.

Following ischemia, brain slices were visualized using fluorescent microscopy techniques to examine the cellular uptake of the labelled nanoparticles. The results showed the presence of fluorescent label in the cells, indicating that metal oxide nanoparticles are efficiently taken up by cells during or after ischemia.

### Example 2

To examine the neuroprotective capability of metal oxide nanoparticles, ceria nanoparticles were added to brain slices following ischemia. In a series of experiments, the nanoparticles were added at two and four hours post-ischemia, and the brain slices were examined for signs of post-ischemic damage and cell death 24 hours after ischemia.

When the nanoparticles were applied two hours after ischemia, the brain tissue showed a significant decrease in cell death when examined 24 hours after ischemia. However, when the ceria was applied four hours after ischemia, the brain tissue did not show any significant decrease in cell death when examined 24 hours after ischemia. This is further evidence that oxidative damage occurs early post-ischemia, and that the production of ROS early in the ischemic injury is responsible for much of the tissue damage measured 24 hours post-ischemia.

### Example 3

As described supra, oxidative and nitrosative damage following ischemic injury are primary contributors to tissue death in the brain. This Example describes the use of a mouse hippocampal brain slice model to test the hypothesis that cerium oxide nanoparticles are neuroprotective in an in-vitro model of stroke. Ceria-based nanoparticles, which readily cross the blood-brain bather (as described in Example 1), neutralize reactive oxygen species by undergoing rapid, reversible reduction/oxidation reactions without generating free radicals in the process.

In brief, transverse brain sections of the hippocampus were prepared from adult CD 1 littermates, and the sections were paired (control versus test) along the rostral-caudal axis. Ischemia was induced by placing the brain slices in a hypoxic, hypoglycemic and acidic aCSF for 30 min after which sections were placed in culture. Nanoparticles (0.2-2 ug/mL, Sigma-Aldrich™) administered during the ischemic insult and present throughout the post-ischemic period, decreased cell death (measured at 24 hours post-ischemia (PI) using a fluorescent, vital exclusion dye) by approximately 50%.

The results show that the neuroprotective effects of ceria-based nanoparticles were apparent as long as the nanoparticles were added within 4 hours post-initiation (“PI”). In non-ischemic controls, ceria nanoparticles did not affect cell viability at the concentrations and over the duration of exposure that were tested. The ceria nanoparticles accumulated in high densities around cellular membranes, mitochondria and neurofilaments in TEM images.

To explore the biological mechanisms of action of ceria, the ischemia-induced accumulation of reactive oxygen species (ROS) in paired brain sections was measured. The results show that ceria decreased ROS production by 32% measured 1 hr PI using the fluorescent probe 5-(and 6-) chloromethyl-2′,7′-dichlorodihydrofluorescein diacetate acetyl ester. Moreover, ceria treatment (1 ug/mL) significantly reduced the ischemia-induced expression of the program cell death protein, apoptosis inducing factor (AIF), in both the nuclear and mitochondrial fractions at 24 hr PI.

These data suggest that cerium oxide nanoparticles mitigate ischemic brain injury by multiple mechanisms and may be a useful therapeutic intervention to reduce oxidative/nitrosative tissue damage.

### Example 4

In another embodiment of the present invention, ceria, mixed ceria/yttria and mixed ceria/platinum nanoparticles are given to an ischemic patient or tissue. The mixed particles are potentially more reactive when applied together and thus would serve as a more potent free radical scavenger. Increased potency would likely result in additional neuroprotective benefits following ischemia.

Thus, the experiments described supra show that application of metal oxide nanoparticles during the period of highest ROS production following the initiation of ischemia, roughly 0-4 hours post-initiation, protects neuronal cells from ischemic injury caused by the increased production of reactive oxygen species.

### Example 5

As described supra, doping metal oxide nanoparticles with rare earth metals can improve or otherwise alter the metal oxide's catalytic function to achive specific therapeutic goals. In these experiments, the in vitro antioxidant efficacy of nanoparticles of ceria, yttrium-doped ceria, and platinum-doped ceria was determined.

The particles were exposed to the superoxide radical, O₂⁻ which was generated by the enzymatic reaction of hypoxanthine in the presence of xanthine oxidase. The extent of inactivation induced by the nanoparticles (1 μg/ml) was determined electrochemically. Results indicated that metal oxides doped with a rare earth metal such as yttrium or with a noble material such as platinum can possess greater antioxidant activity than un-doped metal oxides. When tested in the brain slice model of ischemia, the cerium oxide nanoparticles doped with yttrium showed superior antioxidant activity and a greater reduction in cell killing in the cell compared to cerium oxide alone.

### Example 6

This Example describes the alteration of nanoparticle function or location through modification of one or more of the nanoparticle's surface characteristics. In these experiments, cerium oxide was coated with dextran and applied prior to induction of an ischemic event.

Structural analysis (x-ray diffraction and transmission electron microscopy) of mixed ceria/yttria and mixed ceria/platinum prepared by a precipitation method with dextran indicated the presence of yttria and platinum within the ceria structure. Analysis revealed that the coated nanoparticles were restricted to the extracellular space and that cell sparing following ischemia was reduced compared to uncoated nanoparticles. Since many drugs work by enhancing oxidant activity, dextran-coated nanoparticles might be used to reduce side effects of these agents by reducing the diffusion of the oxidizing agents from the site of desired action. For example, the toxicity of chemotherapeutic agents that work by generating intracellular oxidizing agents might be reduced by surrounding the abnormal cells with dextran coated nanoparticles, which would reduce the diffusion of oxidizing agents into normal tissue. Coatings to enhance cellular uptake can be used increase the specificity of organ targeting.

In addition to dextran, the nanoparticles can be modified with dextran, polyethylene oxide, polyethylene imine, polylactic acid, chitosan, or alginate to tailor surface charge, provide biocompatibility and increase cellular uptake and circulation time in vivo, among other alterations. The size of the nanoparticles may also be varied from ˜1 nm to 100 nm to modify the distribution of the particles and change the antioxidant efficacy of the nanoparticles.

What is claimed is:

1. A metal oxide nanoparticle composition comprising:
a cerium oxide nanoparticle; and
a metal adapted to enhance a neuroprotective activity of said cerium oxide nanoparticle.

2. The metal oxide nanoparticle composition of claim 1, wherein the metal is selected from the group consisting of noble metals and rare earth metals.

3. The metal oxide nanoparticle composition of claim 2, wherein said noble metal is platinum.

4. The metal oxide nanoparticle composition of claim 2, wherein said rare earth metal is selected from the group consisting of gadolinium, samarium, titanium, yttrium, zirconium, and a combination thereof.

5. The metal oxide nanoparticle composition of claim 1, wherein the cerium oxide nanoparticle is approximately 1 nanometer to approximately 100 nanometers in size.

6. A metal oxide nanoparticle composition comprising:
a cerium oxide nanoparticle; and
a surface modifier.

7. The metal oxide nanoparticle composition of claim 6, wherein the surface modifier is selected from the group consisting of polyethylene oxide, polyethylene imine, dextran, polylactic acid, chitosan, alginate, and a combination thereof.

8. The metal oxide nanoparticle composition of claim 6, further comprising a metal adapted to enhance a neuroprotective activity of said cerium oxide nanoparticle.

9. The metal oxide nanoparticle composition of claim 8, wherein the metal is selected from the group consisting of noble metals and rare earth metals.

10. The metal oxide nanoparticle composition of claim 9, wherein said noble metal is platinum.

11. The metal oxide nanoparticle composition of claim 9, wherein said rare earth metal is selected from the group consisting of gadolinium, samarium, titanium, yttrium, zirconium, and a combination thereof.

12. A method of protecting neuronal cells from ischemic injury, comprising the step of:
administering to a subject a metal oxide nanoparticle composition comprising a cerium oxide nanoparticle and a metal adapted to enhance a neuroprotective activity of said cerium oxide nanoparticles.

13. The method of claim 12, wherein the metal is selected from the group consisting of noble metals and rare earth metals.

14. The method of claim 13, wherein said noble metal is platinum.

15. The method of claim 13, wherein said rare earth metal is selected from the group consisting of gadolinium, samarium, titanium, yttrium, zirconium, and a combination thereof

16. The method of claim 12, wherein the metal oxide nanoparticle composition is administered prior to the ischemic injury.

17. The method of claim 16, wherein the metal oxide nanoparticle composition is administered up to about six weeks prior to the ischemic injury.

18. The method of claim 12, wherein the metal oxide nanoparticle composition is administered at a dose of approximately 0.5 μM/kg to approximately 1 μM/kg.

19. A method of protecting neuronal cells from ischemic injury comprising the step of:
administering to a subject a metal oxide nanoparticle composition comprising a cerium oxide nanoparticle and a surface modifier.

20. The method of claim 19, wherein the surface modifier is selected from the group consisting of polyethylene oxide, polyethylene imine, dextran, polylactic acid, chitosan, alginate, and a combination thereof.
